(19) United States
(12) Plant Patent Application Publication
(10) Pub. No.: US 2014/0283234 P1
(43) Pub. Date: Sep. 18, 2014

(54) Blueberry plant named 'C05-190'

(50) ;

(71) Applicants: Florida Foundation Seed Producers, Inc., Marianna, FL (US); CostaExchange Pty Ltd., Corindi (AU)

(72) Inventors: Gary Wright, Corindi Beach (AU); Paul Lyrene, Micanopy, FL (US)

(21) Appl. No.: 13/999,626

(22) Filed: Mar. 13, 2014

Related U.S. Application Data
(60) Provisional application No. 61/851,799, filed on Mar. 13, 2013.

(51) Int. Cl. A01H 5/00

(52) U.S. Cl. PLT/157

(57) ABSTRACT

A new and distinct cultivar of blueberry (Vaccinium corymbosum hybrid) plant named ‘C05-190,’ characterized by its upright plant growth habit, strong growth vigor, low chilling requirement, medium to late time of fruiting, large fruit size, firm fruit suited to handling, good acidity and sweetness balance in the fruit. This combination results in higher quality fruit with a later availability than other varieties.

### LATIN NAME OF THE FAMILY, GENUS, AND SPECIES

Family—Ericaceae

Genus—Vaccinium

Species—corymbosum hybrid

### VARIETY DENOMINATION

The new blueberry plant claimed is of the variety denominated ‘C05-190.’

### BACKGROUND OF THE INVENTION

The present invention relates to a new and distinct perennial variety of Vaccinium corymbosum hybrid (blueberry), which has been given the variety denomination of ‘C05-190.’ The new variety ‘C05-190’ shows distinctive traits such as strong growth vigor, fruit with strong firmness, good acidity and sweetness balance, low chilling requirement. Its market class is that of a fruiting plant. The new variety ‘C05-190’ is intended for use as fresh fruit for shipping, customer pick and processing markets and as a home garden plant.

The new blueberry cultivar is a selection resulting from seedlings produced in a controlled breeding programme of Vaccinium varieties in Florida, USA in 2003 from a cross of the blueberry variety known as ‘Early crisp’ (seed parent) (unpatented) and the blueberry variety known as ‘FL03-061’ (pollen parent) (unpatented). The seed from the cross was sown and grown in Corindi Beach, New South Wales, Australia. The new cultivar was discovered and selected in 2005 as a single plant within a population of seedlings resulted from the controlled cross, in an experimental block in the field at Corindi Beach, New South Wales, Australia, and has since been named ‘C05-190.’ Selection criteria were a combination of strong growth vigor, low chilling requirement, fruit with strong firmness and good acidity and sweetness balance. The new variety was subsequently evaluated for a number of years at the commercial farm at Corindi Beach, New South Wales, Australia.

Asexual reproduction of the new variety ‘C05-190’ by softwood cutting propagation since 2005 at Corindi Beach, New South Wales, Australia has demonstrated that the new variety reproduces true to type with all of the characteristics, as herein described, firmly fixed and retained through successive generations of such asexual propagation, with the clones phenotypically identical to the original plant.

The seed parent ‘Early crisp’ is characterized by an early timing of ripening of fruit. The pollen parent ‘FL03-061’ is characterized by a late to very late timing of ripening of fruit. The new variety ‘C05-190’ differs from the seed and pollen parents in that ‘C05-190’ has a medium to late cropping time, ripening after ‘Early crisp’ and before ‘FL03-061.’ The new variety ‘C05-190’ has maintained its distinguished characteristics throughout successive asexual propagation.

### SUMMARY OF THE INVENTION

The following characteristics of the new variety have been repeatedly observed and can be used to distinguish ‘C05-190’ as a new and distinct variety of Vaccinium corymbosum hybrid:

1. low chilling requirement

2. strong growth vigor

3. upright plant growth habit

4. large fruit size

5. firm fruit

6. good acidity and sweetness balance in the fruit

Plants of the new variety ‘C05-190’ have a medium to late cropping time, which is different from the early cropping of the seed parent ‘Early crisp’ and the late to very late cropping of the pollen parent ‘FL03-061.’ The new variety ‘C05-190’ has maintained its distinguished characteristics through successive asexual propagation.

### BRIEF DESCRIPTION OF THE DRAWINGS

The accompanying photographic illustration shows typical specimens in full color of the foliage and fruit of the new variety ‘C05-190.’ The colors are as nearly true as is reasonably possible in a color representation of this type.

FIG. 1 is a photograph of the new variety ‘C05-190, ’ showing parts of the plant in comparison with other varieties.

FIG. 2 is a photograph of the flowers of the new variety ‘C05-190.’

### DETAILED BOTANICAL DESCRIPTION

The following detailed description sets forth the distinctive characteristics of ‘C05-190.’ The data which defines these characteristics was collected from asexual reproductions of the original selection. Dimensions, sizes, colors, and other characteristics are approximations and averages set forth as accurately as possible. The plant history was taken on plants approximately 4 years of age, and the descriptions relate to plants grown in the field in Corindi Beach, New South Wales, Australia. Descriptions of fruit characteristics were made on fruit grown in Corindi Beach, New South Wales, Australia. Color designations are from RHS Chart—edition 2007.
Classification:
   Family.—Ericaceae.
   Genus.—Vaccinium.
   Species.—corymbosum hybrid.
   Common name.—blueberry.
Parentage:
   Seed parent.—‘Early crisp’ (unpatented).
   Pollen parent.—‘FL03-061’ (unpatented).
   Market class.—Suitable for the Commercial, Hand Harvest Fresh, Processed, and Home Garden Markets.
Plant:
   Parentage.—‘Early crisp’×‘FL03-061’.
   Chilling requirement.—Low chilling.
Foliage:
   Leaf length.—Average 67 mm (range 53-75 mm).
   Leaf width.—Average 36 mm (range 29-40 mm).
   Leaf nectarines.—Absent.
Flowers:
   Time of beginning of flowering.—Early to medium.
   Time of 50% Anthesis.—25^{th} June.
   Corolla.—a. Color: White b. Width: Average 5.2mm (range 4-7mm) c. Anthocyanin Coloration of Corolla: Absent or very weak.
Fruit:
   Time of fruit ripening.—Medium to late.
   Time of 50% Maturity.—1^{st} September.
   Berry surface wax abundance.—Medium to low.
   Berry weight.—Average 2.9 g (range 2.7-3.2 g).

1. A new and distinct variety of blueberry plant named ‘C05-190,’ substantially as illustrated and described herein.
